(19) United States
(12) Patent Application Publication
(10) Pub. No.: US 2021/0015583 A1
(43) Pub. Date: Jan. 21, 2021

(54) AUGMENTED REALITY SYSTEM AND METHOD FOR TELE-PROCTORING A SURGICAL PROCEDURE

(71) Applicant: Surgical Theater, Inc., Mayfield Village, OH (US)

(72) Inventors: MORDECHAI AVISAR, HIGHLAND HEIGHTS, OH (US); Alon Yakob Geri, Orange Village, OH (US); Nate Regev, Beverly Hills, CA (US)

(21) Appl. No.: 16/930,222

(22) Filed: Jul. 15, 2020

Related U.S. Application Data
(60) Provisional application No. 62/874,315, filed on Jul. 15, 2019.

(51) Int. Cl. A61B 90/00; G06F 3/14; G06T 19/00; H04N 7/08; H04N 5/232

(52) U.S. Cl. CPC A61B 90/36; G06F 3/14; G06T 19/006; H04N 7/0806; A61B 2090/502; G06T 2210/41; G06T 2219/024; A61B 2090/365; H04N 5/23296

(57) ABSTRACT

A system for tele-proctoring a surgical procedure includes an augmented reality head mounted display and a computer. The computer is configured to receive a visual experienced from the eyes of an onsite physician via the augmented reality head mounted display; receive additional content experienced by the onsite physician via the augmented reality head mounted display; integrate the visual experienced from the eyes of an onsite physician and the additional content into a single integrated view experienced by the onsite physician; communicate the integrated view to a remote computer for display on a remote display; receive an interaction with the integrated view from a remote physician via the remote computer; and present the interaction to the onsite physician via the augmented reality head mounted display.

### CROSS-REFERENCE TO RELATED APPLICATION

This application claims priority from U.S. provisional patent application Ser. No. 62/874,315 filed on Jul. 15, 2019 which is incorporated by reference herein in its entirety.

### FIELD OF DICSLOURE

The present disclosure relates to the field of surgical procedures and more specifically to the field of tele-proctoring a surgical procedure.

### BACKGROUND

Surgical procedures may be complex, the success of which may be crucial to a patient's well-being. Thus, in order to perform surgical procedures, a physician is commonly required to undergo extensive training including performing or participating in surgical procedures under the guidance and supervision of a senior and more experienced physician. And even beyond the initial training and guidance, a senior physician may be required to proctor a surgical procedure being performed by a less senior physician and to confirm certain maneuvers, decisions, or techniques being selected or performed by the less senior physician. For example, a surgical procedure involving a craniotomy may require a senior physician to approve a marking made by a less senior physician indicative of where the procedure is to be performed before the actual procedure is initiated.

With advances in and increasing availability of various communication technologies, tele-proctoring or tele-assisting is becoming an increasingly popular option individuals or teams to remotely provide training, guidance, and support to other individuals or teams from a remote location. Moreover, augmented reality technologies are increasingly being used to facilitate remote interactions between two individuals by enabling remote individuals to overlay instructions on top of real-world views for local users. For example, augmented reality technology such as Microsoft's Dynamic 365 Remote Assist may enable such remote interaction. However, using such augmented reality technologies specifically for tele-proctoring a surgical procedure may not be possible or practical because of specific environmental conditions present within an operating room.

### SUMMARY

A system for tele-proctoring a surgical procedure includes an augmented reality head mounted display and a computer, including one or more processors, one or more computer-readable tangible storage devices, and program instructions stored on at least one of the one or more storage devices for execution by at least one of the one or more processors. The program instructions are configured to receive a visual experienced from the eyes of an onsite physician via the augmented reality head mounted display; receive additional content experienced by the onsite physician via the augmented reality head mounted display; integrate the visual experienced from the eyes of an onsite physician and the additional content into a single integrated view experienced by the onsite physician; communicate the integrated view to a remote computer for display on a remote display; receive an interaction with the integrated view from a remote physician via the remote computer; and present the interaction to the onsite physician via the augmented reality head mounted display.

A method for tele-proctoring a surgical procedure includes the steps of receiving a visual experienced from the eyes of an onsite physician via an augmented reality head mounted display; receiving additional content experienced by the onsite physician via the augmented reality head mounted display; integrating the visual experienced from the eyes of an onsite physician and the additional content into a single integrated view experienced by the onsite physician; communicating the integrated view to a remote computer for display on a remote display; receiving an interaction with the integrated view from a remote physician via the remote computer; and presenting the interaction to the onsite physician via the augmented reality head mounted display.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example augmented reality tele-proctoring system.

FIG. 2 illustrates an example augmented reality tele-proctoring system.

FIG. 3 illustrates an example augmented reality tele-proctoring system.

FIG. 4 illustrates an example augmented reality tele-proctoring system.

FIG. 5 illustrates an example method for tele-proctoring a surgical procedure.

FIG. 6 illustrates an example computer implementing the example augmented reality tele-proctoring systems of FIGS. 1-4.

### DETAILED DESCRIPTION

The following acronyms and definitions will aid in understanding the detailed description:

AR—Augmented Reality—A live view of a physical, real-world environment whose elements have been enhanced by computer generated sensory elements such as sound, video, or graphics.

VR—Virtual Reality—A 3Dimensional computer generated environment which can be explored and interacted with by a person in varying degrees.

HMD—Head Mounted Display refers to a headset which can be used in AR or VR environments. It may be wired or wireless. It may also include one or more add-ons such as headphones, microphone, HD camera, infrared camera, hand trackers, positional trackers etc.

Controller—A device which includes buttons and a direction controller. It may be wired or wireless. Examples of this device are Xbox gamepad, PlayStation gamepad, Oculus touch, etc.

SNAP Model—A SNAP case refers to a 3D texture or 3D objects created using one or more scans of a patient (CT, MR, fMR, DTI, etc.) in DICOM file format. It also includes different presets of segmentation for filtering specific ranges and coloring others in the 3D texture. It may also include 3D objects placed in the scene including 3D shapes to mark specific points or anatomy of interest, 3D Labels, 3D Measurement markers, 3D Arrows for guidance, and 3D surgical tools. Surgical tools and devices have been modeled for education and patient specific rehearsal, particularly for appropriately sizing aneurysm clips.

Avatar—An avatar represents a user inside the virtual environment.

MD6DM—Multi Dimension full spherical virtual reality, 6 Degrees of Freedom Model. It provides a graphical simulation environment which enables the physician to experience, plan, perform, and navigate the intervention in full spherical virtual reality environment.

A surgery rehearsal and preparation tool previously described in U.S. patent application No. 8,311,791, incorporated in this application by reference, has been developed to convert static CT and MRI medical images into dynamic and interactive multi-dimensional full spherical virtual reality, six (6) degrees of freedom models (“MD6DM”) based on a prebuilt SNAP model that can be used by physicians to simulate medical procedures in real time. The MD6DM provides a graphical simulation environment which enables the physician to experience, plan, perform, and navigate the intervention in full spherical virtual reality environment. In particular, the MD6DM gives the surgeon the capability to navigate using a unique multidimensional model, built from traditional two-dimensional patient medical scans, that gives spherical virtual reality 6 degrees of freedom (i.e. linear; x, y, z, and angular, yaw, pitch, roll) in the entire volumetric spherical virtual reality model.

The MD6DM is rendered in real time using a SNAP model built from the patient's own data set of medical images including CT, MRI, DTI etc., and is patient specific. A representative brain model, such as Atlas data, can be integrated to create a partially patient specific model if the surgeon so desires. The model gives a 360° spherical view from any point on the MD6DM. Using the MD6DM, the viewer is positioned virtually inside the anatomy and can look and observe both anatomical and pathological structures as if he were standing inside the patient's body. The viewer can look up, down, over the shoulders etc., and will see native structures in relation to each other, exactly as they are found in the patient. Spatial relationships between internal structures are preserved and can be appreciated using the MD6DM.

The algorithm of the MD6DM takes the medical image information and builds it into a spherical model, a complete continuous real time model that can be viewed from any angle while “flying” inside the anatomical structure. In particular, after the CT, MRI, etc. takes a real organism and deconstructs it into hundreds of thin slices built from thousands of points, the MD6DM reverts it to a 3D model by representing a 360° view of each of those points from both the inside and outside.

Described herein is an augmented reality (“AR”) system, leveraging a MD6DM model, for tele-proctoring a surgical procedure. In particular, the AR system enables a remotely located physician to interact with and proctor a surgical procedure being performed on a patient by an onsite physician by providing the remote physician the same view as being experienced by the onsite physician via an augmented reality headset, the view including a visual experienced from the eyes of the onsite physician as well as additionally integrated content such as a prebuilt MD6DM model, and providing the remote physician with means for interacting with the view such that the onsite physician experiences the interactions. Thus, the patient is provided with the care and expertise that may not otherwise be available due to location and availability of healthcare professionals at the onsite location.

Integrating the additional content and features into the example AR systems as will be described herein in more detail allows for increased comfort for surgeons as well increased adoption since the AR HIVID may be worn during the entire surgical procedure without needing to take it off in order to view a microscope, to put on other loupe, and so on. The system described herein also enables better multitasking for a physician. Finally, it enables a remote attending physician to be more involved in a surgical procedure and thereby increase the safety of the procedure and reduce risk of error during the procedure.

It should be appreciated that example systems described herein may be used for pre-operative planning, preparing in the operating room, and during an actual surgical procedure. It should be further appreciated that, although an example application for use during a craniotomy may be described herein, the example systems may be sued for any suitable surgical procedure.

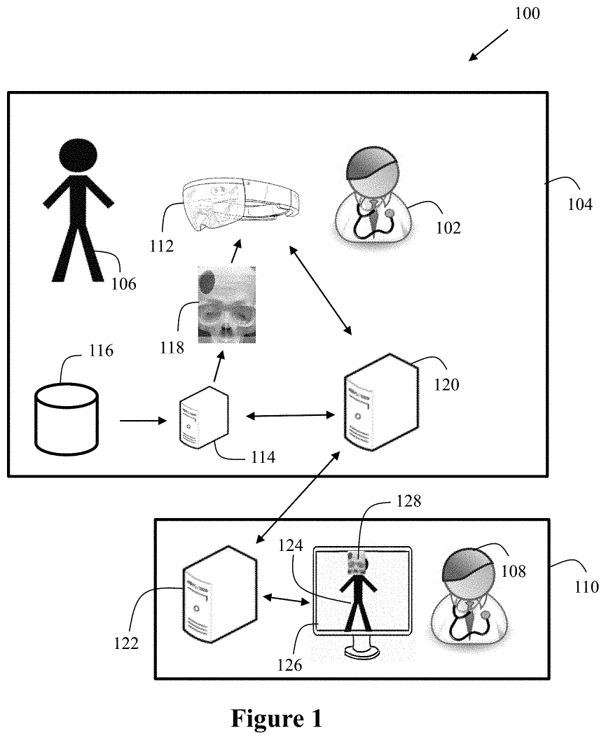

FIG. 1 illustrates an AR tele-proctoring system 100 for enabling an onsite physician 102 located in a hospital 104 (or any similar suitable location) and performing a surgical procedure on a patient 106 to communicate with and interact with a remote physician 108 located in a remote location 110. In particular, the AR system 100 enables the remote physician 108 to proctor and assist with the surgical procedure from the remote location 110. Proctoring a surgical procedure can mean, for example, answering questions during the surgical procedure, making suggestions or providing instructions about how to perform the procedure, and confirming that the actions being taken by the onsite physician 102 are accurate and correct. It should be appreciated that, although the AR system 100 is described as being used during a surgical procedure, the AR system 100 can also be used for pre-operative planning and preparation.

The AR system 100 includes an AR head mounted display (“HMD”) 112 for providing the onsite physician 102 with an AR view including a live real life visual of the patient 106 in combination with additionally integrated content. For example, the AR system 100 includes an MD6DM computer 114 for retrieving a SNAP model from a SNAP database 116, for rendering a MD6DM model 118, and for providing the MD6DM model 118 to the AR HIVID 112. The MD6DM computer 114, in combination with the AR HIVID 112, is configured to synchronize the MD6DM model with and overlay it on top of the live real life visual of the patient 106 in order to create an AR view (not shown) of the patient 106 via the AR HIVID 112.

The AR system 100 further includes a tele-computer 120 configured to communicate to a remote computer 122 the AR view experienced by the onsite physician 102. In particular, the tele-computer 120 is configured to receive a live video feed from a camera on the AR HIVID 112 that captures and represents the live real life visual of the patient 106 as seen by the onsite physician 102. The tele-computer 120 is further configured to receive additionally integrated content and to synchronize the additionally integrated content with the live video feed from the AR HIVID 112. For example, the tele-computer 120 is configured to receive from the MD6DM computer 114 the rendered MD6DM model 118 and to synchronize the MD6DM model 118 with the live video feed.

The remote computer 122 is configured to communicate the AR view including a live video feed 124 of the patient and a remote MD6DM model 128 synchronized with the live video feed 124 to a remote display 126. It should be appreciated that the remote display 126 can be any suitable type of display, including a head mounted display (not shown). Thus, the remote physician 108 is able to experience in real time vie the remote display 126 the same view, including the live real life visual of the patient 106 and the additionally integrated content, as being experienced by the onsite physician 102.

In one example, the remote location 110 includes a remote integrated content computer such as an MD6DM computer (not shown) for retrieving the additionally integrate content such as the SNAP model from a remote database (not shown). Thus, the tele-computer 122 does not need to synchronize or integrate any additional content with the live video feed received from the AR HMD 112. Instead, the tele-computer 122 communicates the live video feed to the remote computer 122 without additional content, thereby conserving communication bandwidth. In such an example, the remote computer 122 retrieves the additional content from the remote integrated content computer and performs the integration and synchronization with the live video feed at the remote location 110. For example, the remote computer 122 is configured to retrieve a SNAP model from a remote SNAP database (not shown) and render the remote MD6DM model 128. The remote computer 122 is further configured to synchronize the remote MD6DM model 128 with the live video feed 124 and integrate the two onto the remote display 126 to form the view representative of the same view being experienced by the onsite physician 102.

The remote computer 122 is further configured to receive, via either the display 126 or via additional peripheral input devices (not shown), interactions with the view from the remote physician 108. For example, remote computer 122 may receive from the remote physician 108 markups, notes, and other suitable input interactions with both the live video feed 124 of the patient and the additionally integrated and synchronized content such as the remote MD6DM model 128. The interactions may include, for example, the remote physician 108 manipulating the remote MD6DM model 128 or placing a mark on the remote MD6DM model 128 to indicate where to make an incision. The remote computer 122 is further able to distinguish between interactions with the live video feed 124 and interactions with the additionally integrated content such as the remote MD6DM model 128.

The remote computer 122 is further configured to communicate the remote interactions of the remote physician 108 to tele-computer 120, which in turn is configured to communicate and to appropriately render the received remote interactions to the AR HIVID 112 in connection with the corresponding content. The remote tele-computer 120 is configured to render received remote interactions with the respective content based on the distinctions identified between the interactions. For example, the tele-computer 120 may be configured to synchronize and integrate with the MD6DM model 118 the received remote interactions with the remote MD6DM model 128 such that the onsite physician 102 is able to experience the marked view in the MD6DM model 118 as provided by the remote physician 108. In another example, the MD6DM computer 114 may be configured to receive the interactions from tele-computer 120 and to synchronize and integrate the remote interactions with the MD6DM model 118. It should be appreciated that, although the MD6DM computer 114 and the tele-computer 120 are described as two distinct computers, the MD6DM computer 114 and the tele-computer 120 may be combined into a single computer (not illustrated).

By communicating markings and other suitable interactions from the remote physician 108 to the onsite physician 102, the AR system 100 is able to facilitate proctoring of a craniotomy, for example, which requires a physician to mark an entry point for where the procedures should be initiated. Providing such a mark isn't always feasible or practical based on a real life live view alone and often requires the aid of additionally integrated content such a MD6DM model over-lay on top of the skull. Providing a remote view that includes the additionally integrated content enables improved proctoring capabilities and collaboration since the onsite physician 102 and the remote physician 108 are able to interact with each other and provide real time feedback with respect to both the real life live view as well as the additionally integrated content.

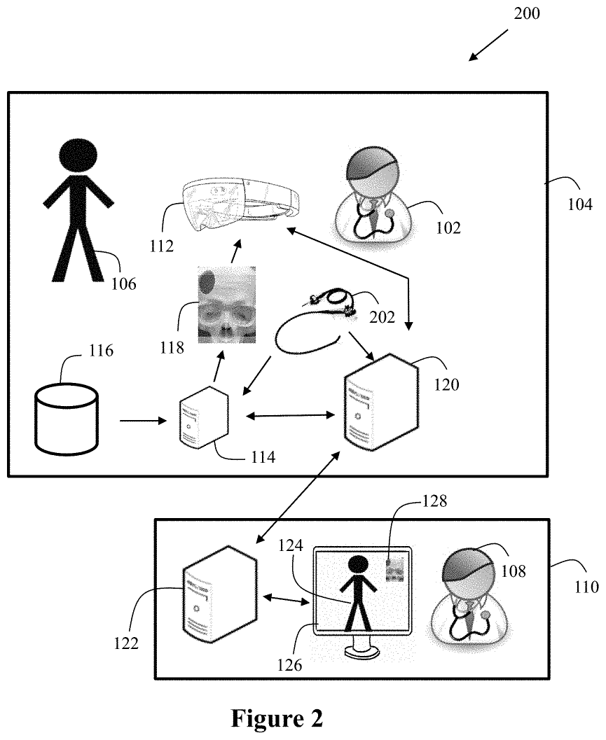

In one example AR system 200, as illustrated in FIG. 2, the additional content integrated with the live real life visual of the patient 106 and included in the view experienced by the onsite physician includes video generated by an endoscope 202. For example, an onsite physician 102 may use an endoscope to obtain a closeup inside view of the patient 106. The closeup view from the endoscope 202 is incorporated into the view experienced by the onsite physician 102 via the AR HIVID 112. For example, the closeup view from the endoscope may be presented to the onsite physician 102 in a portion of a lens of the AR HMD 112 such that the onsite physician 102 may easily look back and forth between the real life live view the patient or the closeup view form the endoscope, all within the same AR HMD 112.

Thus, in order for the remote physician 108 to experience the same view as the onsite physician 102, the tele-computer 120 is further configured to communicate to the remote computer 122 the same video generated by the endoscope 202 in addition to the live video feed captured by the camera on the AR HIVID 112. In one example, the remote computer 122 is configured to integrate the additional content (e.g. the video generated by the endoscope 202) with the live video feed from the camera on the AR HIVID 112 to produce on the display 126 the same integrated view for the remote physician 108 as experienced by the onsite physician 102. Specifically, the remote computer 122 may present on the screen 126 simultaneously both the real life view received from the camera on the HIVID 112 in a first portion of the display 126 as well as the closeup view received form the endoscope 202 in a second portion of the display 126.

In another example, the remote computer 122 may be configured to display one of either the real life view received from the camera on the HIVID 112 or the closeup view received form the endoscope 202, depending on a selection of the remote physician 108 via an interface provided by the remote computer 122. For example, the remote physician may selectively toggle between seeing the real life view received from the camera on the HIVID 112 or the closeup view received form the endoscope 202 and selectively interact with either one at any time. In another example, the remote computer 122 may be configured to automatically toggle the display between either the real life view received from the camera on the HIVID 112 or the closeup view received form the endoscope 202 depending on action taken by the onsite physician 102. For example, the AR HIVID 112 may be configured to track the eye movement of the onsite physician 102. In particular, the AR HIVID 112 may be configured to determine when the onsite physician's 102 eyes are focused on closeup view presented in the AR view and when the onsite physician's 102 eyes are focused anywhere else within the AR view. Thus, based on the onsite physician's 102 eye focus, the remote computer 122 may be configured to automatically present to the display 126 the same corresponding view.

In one example, the MD6DM computer 114 may be configured to receive the closeup video feed from the endoscope 202 and to synchronize and overlay a closeup view of the MD6DM model 118 over the closeup video feed before communicating the combined integrated closeup video feed to the AR HMD 112. In such an example, the tele-computer 122 may be configured to provide the remote physician 108 with the same view experienced by the onsite physician 102, including an integrated and synchronized closeup view with an MD6DM overlay generated from the endoscope 202 as well as the real life live view received from the camera on the AR HMD 112.

As previously described, the remote computer 114 is configured to receive and distinguish different types of interactions with the view from the remote physician 108 and communicate the distinguished interactions to the tele-computer 120, which in turn appropriately renders the interactions in connection with the corresponding content.

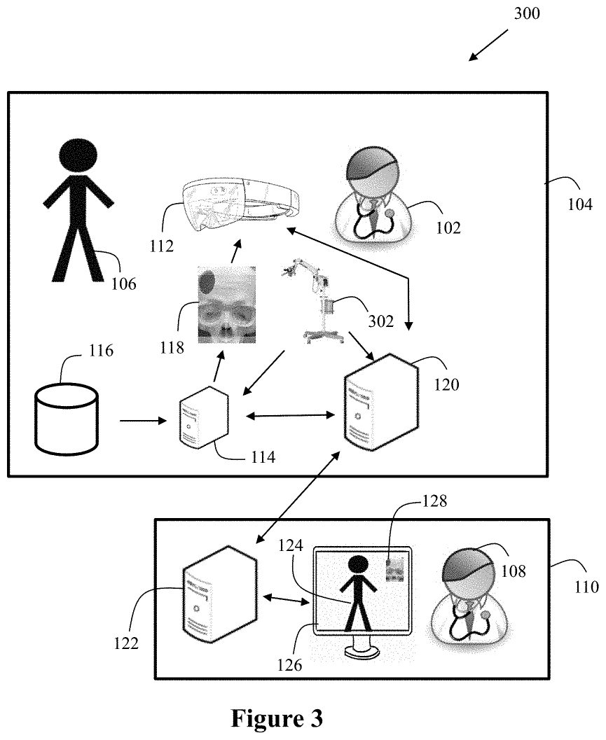

In one example AR system 300, as illustrated in FIG. 3, the additional content integrated with the live real life visual of the patient 106 and included in the view experienced by the onsite physician includes video generated by a microscope 302. The video generated by the microscope 302 may or may not be synchronized with an MD6DM model, as described in the previous example. Also as in the previous example, the video generated by the microscope 302, either with or without MD6DM integration, may be presented to and experienced by the onsite physician 102 in an augmented view via the AR HMD 112. Similarly as described for the previous example of the endoscope video, the additional content of the video from the microscope 302 may be presented to the remote physician 108 as part of the experienced view.

In one example, rather than providing a video feed from the microscope 302 into the AR view via the AR HIVID 112, the onsite physician may choose to consume the microscope view by interacting directly with the microscope. For example, the onsite physician 102 may look through a viewer on the microscope 302 in order to see a closeup view of the patient 106. However, the onsite physician 102 may still be wearing the AR HMD 112 and intend for the remote physician 108 to experience the same view. Thus, in such an example, a video feed from the microscope 302 may still be provided to by the tele-computer 120 to the remote computer 122 in order to enable the remote computer 122 to generate the same view for the remote physician 108 as experienced by the onsite physician 102.

As described in the previous example, the remote computer 122 may be configured to display one of either the real life view received from the camera on the HIVID 112 or the closeup view received form the microscope 302, depending on a selection of the remote physician 108 via an interface provided by the remote computer 122.

In another example, the remote computer 122 may be configured to automatically toggle the display between either the real life view received from the camera on the HIVID 112 or the closeup view received form the microscope 302 depending on action taken by the onsite physician 102. For example, the tele-computer 120 may be configured to determine, based on motion sensors or other suitable types of sensors on AR HIVID 112 and based on the video received from the AR HIVID 112, the head position/location of the onsite physician 102. In particular, when it is determined that the onsite physician's 102 head is tilted over top of or otherwise positioned at or near a viewer of the microscope 302, the remote computer 122 may be configured to automatically present to the display 126 the view feed from the microscope and to present the real life video feed from AR HIVID 112 when the onsite physician's 102 head is positioned otherwise.

In one example, the MD6DM computer 114 may be configured to receive the closeup video feed from the microscope 302 and to synchronize and overlay a closeup view of the MD6DM model 118 over the closeup video feed before communicating the combined integrated closeup video feed to the AR HIVID 112. In another example, the MD6DM computer 114 may be configured to inject, synchronize, and overlay a closeup view of the MD6DM model 118 directly into the view experienced by the looking into the view finder of the microscope 302. In such examples, the tele-computer 122 may be configured to provide the remote physician 108 with the same view experienced by the onsite physician 102, including an integrated and synchronized closeup view with an MD6DM overlay generated from the microscope 302 as well as the real life live view received from the camera on the AR HIVID 112.

As previously described, the remote computer 114 is configured to receive and distinguish different types of interactions with the view from the remote physician 108 and communicate the distinguished interactions to the tele-computer 120, which in turn appropriately renders the interactions in connection with the corresponding content.

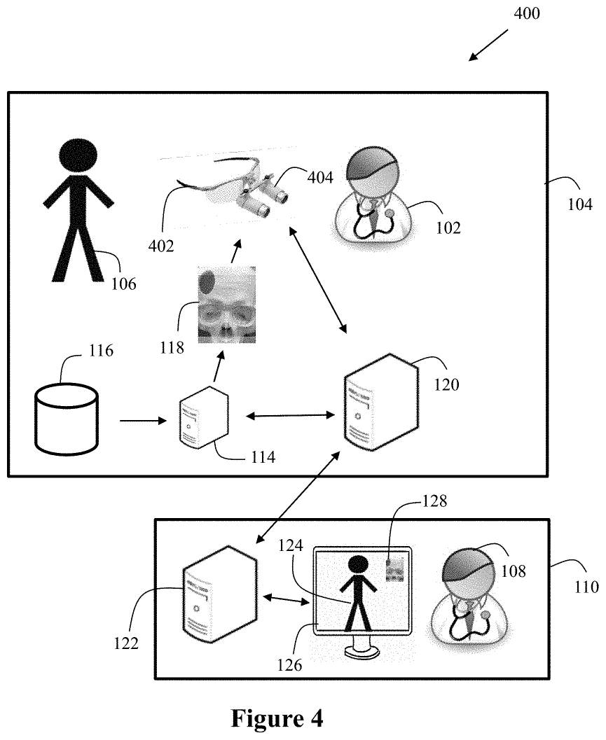

In one example, as illustrated in FIG. 4, an AR HIVID 402 may have integrated (or removeable/detachable) loupes 404 for enabling the onsite physician 102 to get a closeup view of the patient 106. For example, the onsite physician 102 may look straight through the AR HIVID 402 in order to experience a real life live view of the patient 106. The onsite physician 102 may also choose to look through the loupes 404 at any time in order to get a closeup view. Thus, in order to provide the remote physician 108 with the same experience as viewed by the onsite physician, the zoom level of the live video received from the AR HIVID 402 and provided to the remote computer 122 is adjusted according to the onsite physician's 102 eye position relative to the loupes 404 determined by the AR HIVID 402. In particular, if the AR HIVID 402 determines that the onsite physician's eyes are looking through the loupes 404, then the live video received by the camera on the AR HIVID 402 and provided to the remote computer 122 is zoomed in to a closer view based on the magnification strength of the loupes 404. In one example, the camera on the AR HIVID 402 is configured to automatically zoom in based on the determined eye position. In another example, the tele-computer 120 is configured to adjust the live video received from the camera of AR HIVID 402 based on the determined eye position.

In on example, the real life live view experienced via the AR HMD 402 may be augmented by a synchronized MD6DM model. In such an example, the MD6DM model may be adjusted and zoomed in as appropriate, depending on the eye position of the onsite physician 102. Similarly, the real life live video received from the camera on the AR HIVID 402 and provided to the remote computer 122 may synchronized with an MD6DM model, either at the remote site 110 or at the hospital 104 before communicated to the remote site 110. In addition, the MD6DM model synchronized with the real life live video for presentation at the remote site 110 may also be zoomed, depending on the onsite physician's 102 eye position.

As previously described, the remote computer 114 is configured to receive and distinguish different types of interactions with the view from the remote physician 108 and communicate the distinguished interactions to the tele-computer 120, which in turn appropriately renders the interactions in connection with the corresponding content.

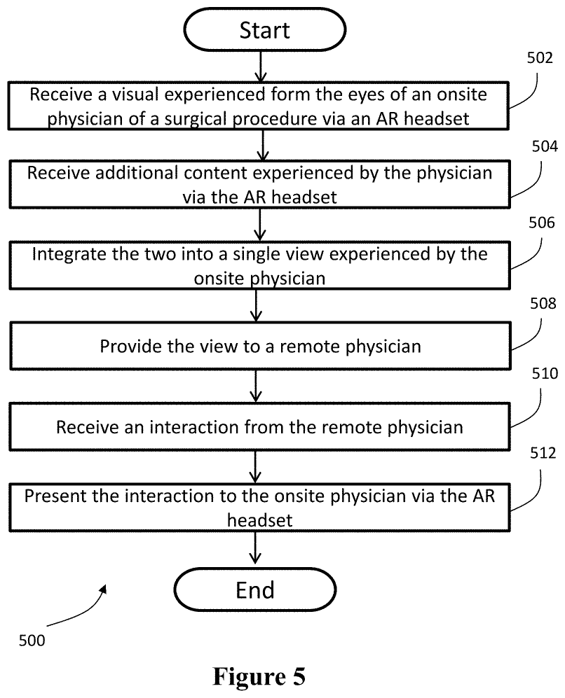

FIG. 5 illustrates an example method for tele-proctoring a surgical procedure. At 502, a visual experienced form the eyes of an onsite physician of a surgical procedure via an AR headset is received. At 504, additional content experienced by the onsite physician via the AR headset is received. At 506, the visual experienced form the eyes of an onsite physician and the additional content is integrated into a single view experienced by the onsite physician. At 508, the view is provided to a remote physician. At 510, an interaction from the remote physician is received. At 512, the interaction is presented to the onsite physician via the AR headset.

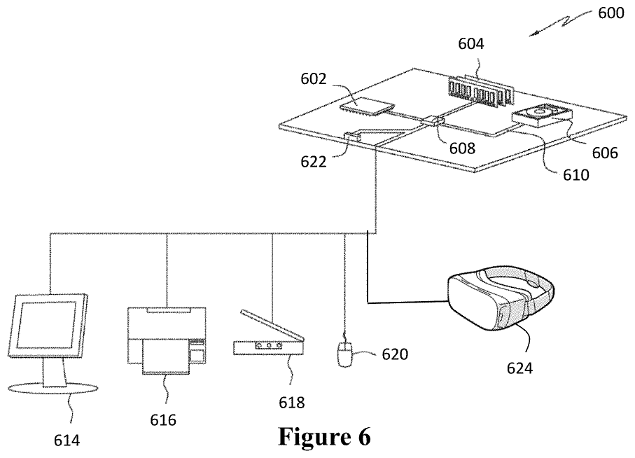

FIG. 6 is a schematic diagram of an example computer for implementing the tele-computer 114, the MD6DM computer 116, and the remote computer 122 of FIG. 1. The example computer 600 is intended to represent various forms of digital computers, including laptops, desktops, handheld computers, tablet computers, smartphones, servers, and other similar types of computing devices. Computer 600 includes a processor 602, memory 604, a storage device 606, and a communication port 608, operably connected by an interface 610 via a bus 612.

Processor 602 processes instructions, via memory 604, for execution within computer 600. In an example embodiment, multiple processors along with multiple memories may be used.

Memory 604 may be volatile memory or non-volatile memory. Memory 604 may be a computer-readable medium, such as a magnetic disk or optical disk. Storage device 606 may be a computer-readable medium, such as floppy disk devices, a hard disk device, optical disk device, a tape device, a flash memory, phase change memory, or other similar solid state memory device, or an array of devices, including devices in a storage area network of other configurations. A computer program product can be tangibly embodied in a computer readable medium such as memory 604 or storage device 606.

Computer 600 can be coupled to one or more input and output devices such as a display 614, a printer 616, a scanner 618, a mouse 620, and a HMD 624.

As will be appreciated by one of skill in the art, the example embodiments may be actualized as, or may generally utilize, a method, system, computer program product, or a combination of the foregoing. Accordingly, any of the embodiments may take the form of specialized software comprising executable instructions stored in a storage device for execution on computer hardware, where the software can be stored on a computer-usable storage medium having computer-usable program code embodied in the medium.

Databases may be implemented using commercially available computer applications, such as open source solutions such as MySQL, or closed solutions like Microsoft SQL that may operate on the disclosed servers or on additional computer servers. Databases may utilize relational or object oriented paradigms for storing data, models, and model parameters that are used for the example embodiments disclosed above. Such databases may be customized using known database programming techniques for specialized applicability as disclosed herein.

Any suitable computer usable (computer readable) medium may be utilized for storing the software comprising the executable instructions. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read -only memory (ROM), an erasable programmable read-only memory (EPROM or Flash memory), a compact disc read-only memory (CDROM), or other tangible optical or magnetic storage device; or transmission media such as those supporting the Internet or an intranet.

In the context of this document, a computer usable or computer readable medium may be any medium that can contain, store, communicate, propagate, or transport the program instructions for use by, or in connection with, the instruction execution system, platform, apparatus, or device, which can include any suitable computer (or computer system) including one or more programmable or dedicated processor/controller(s). The computer usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, local communication busses, radio frequency (RF) or other means.

Computer program code having executable instructions for carrying out operations of the example embodiments may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perl, Smalltalk, C++, C#, Object Pascal, or the like, artificial intelligence languages such as Prolog, a real-time embedded language such as Ada, or even more direct or simplified programming using ladder logic, an Assembler language, or directly programming using an appropriate machine language.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

1. A system for tele-proctoring a surgical procedure, comprising:
an augmented reality head mounted display; and
a computer comprising one or more processors, one or more computer-readable tangible storage devices, and program instructions stored on at least one of the one or more storage devices for execution by at least one of the one or more processors, the program instructions being configured to:
receive a visual experienced from the eyes of an onsite physician via the augmented reality head mounted display;
receive additional content experienced by the onsite physician via the augmented reality head mounted display;
integrate the visual experienced from the eyes of an onsite physician and the additional content into a single integrated view experienced by the onsite physician;
communicate the integrated view to a remote computer for display on a remote display;
receive an interaction with the integrated view from a remote physician via the remote computer; and
present the interaction to the onsite physician via the augmented reality head mounted display.

2. The system of claim 1, wherein the additional content comprises a dynamic and interactive multi-dimensional anatomical model, and wherein the computer is configured to integrate the visual experienced and the additional content by synchronizing and overlaying the anatomical model with a real visual of an anatomy of a patient experienced via the augmented reality head mounted display.

3. The system of claim 1, wherein the augmented reality head mounted display comprises a camera configured to capture a live video feed representative of a live real life visual of a patient anatomy as experienced by the onsite physician, and wherein the computer is configured to communicate the live video feed and the additional content to the remote computer.

4. The system of claim 1, wherein the interaction comprises at least one of a markup and a note, the interaction being indicative of an instruction for performing a surgical procedure.

5. The system of claim 1, wherein the computer is configured to distinguish an interaction with the visual experienced from the eyes of an onsite physician and an interaction with the additional content for identifying a distinction.

6. The system of claim 5, wherein the computer is configured to render the received interaction based on the identified distinction.

7. The system of claim 3, wherein the additional content comprises close-up video generated by an endoscope, and wherein the computer is configured to communicate the live video feed and the close-up video to the remote computer.

8. The system of claim 3, wherein the augmented reality head mounted display comprises magnifying loupes for enabling a close-up view via the augmented reality head mounted display, wherein the augmented reality head mounted display is configured to determine eye position of the onsite physician relative to the loupes, and wherein the computer is configured to adjust a zoom level of the live video feed communicated to the remote computer based the determined eye position.

9. A method for tele-proctoring a surgical procedure, comprising:
receiving a visual experienced from the eyes of an onsite physician via an augmented reality head mounted display;
receiving additional content experienced by the onsite physician via the augmented reality head mounted display;
integrating the visual experienced from the eyes of an onsite physician and the additional content into a single integrated view experienced by the onsite physician;
communicating the integrated view to a remote computer for display on a remote display;
receiving an interaction with the integrated view from a remote physician via the remote computer; and
presenting the interaction to the onsite physician via the augmented reality head mounted display.

10. The method of claim 9, wherein the additional content comprises a dynamic and interactive multi-dimensional anatomical model, and wherein the step of integrating the visual experienced and the additional content comprises the step of synchronizing and overlaying the anatomical model with a real visual of an anatomy of a patient experienced via the augmented reality head mounted display.

11. The method of claim 9, wherein communicating the integrated view comprises communicating additional content integrated with a live video feed representative of a live real life visual of a patient anatomy as experienced by the onsite physician captured by a camera on the augmented reality head mounted display.

12. The method of claim 9, wherein the interaction comprises at least one of a markup and a note, the interaction being indicative of an instruction for performing a surgical procedure.

13. The method of claim 9, further comprising the step of distinguishing an interaction with the visual experienced from the eyes of an onsite physician and an interaction with the additional content for identifying a distinction.

14. The method of claim 13, wherein the step of presenting the interaction includes the step of rendering the received interaction based on the identified distinction.

15. The method of claim 11, wherein the additional content comprises close-up video generated by an endoscope, and wherein the step of communicating the integrated view includes the step of communicating the live video feed and the close-up video to the remote computer.

16. The system of claim 11, further comprising determining eye position of the onsite physician relative to loupes disposed on the augmented reality head mounted display, and wherein communicating the integrated view comprises adjusting a zoom level of the live video feed communicated to the remote computer based the determined eye position.

17. A method for tele-proctoring a surgical procedure, comprising:
receiving a visual experienced from the eyes of an onsite physician via an augmented reality head mounted display;
receiving additional content including a dynamic and interactive multi-dimensional anatomical model experienced by the onsite physician via the augmented reality head mounted display;
integrating the visual experienced from the eyes of an onsite physician and the additional content including synchronizing and overlaying the anatomical model with a real visual of an anatomy of a patient experienced via the augmented reality head mounted display into a single integrated view experienced by the onsite physician;
determining eye position of the onsite physician relative to loupes disposed on the augmented reality head mounted display;
communicating the integrated view to a remote computer for display on a remote display by communicating additional content integrated with a live video feed representative of a live real life visual of a patient anatomy as experienced by the onsite physician captured by a camera on the augmented reality head mounted display and by adjusting a zoom level of the live video feed communicated to the remote computer based the determined eye position;
distinguishing an interaction with the visual experienced from the eyes of an onsite physician and an interaction with the additional content for identifying a distinction;
receiving an interaction with the integrated view from a remote physician via the remote computer; and
presenting the interaction to the onsite physician via the augmented reality head mounted display by rendering the received interaction based on the identified distinction.

18. The method of claim 17, wherein the additional content further comprises close-up video generated by an endoscope, and wherein the step of communicating the integrated view includes the step of communicating the live video feed and the close-up video to the remote computer.

19. The method of claim 17, wherein the interaction comprises at least one of a markup and a note, the interaction being indicative of an instruction for performing a surgical procedure.
